(19) United States
(12) Patent Application Publication
(10) Pub. No.: US 2005/0136080 A1
(43) Pub. Date: Jun. 23, 2005

(54) Cosmetic solid lead

(75) Inventors: Reiner Appel, Oberasbach (DE); Martin Haller, Nurnberg (DE); Gerhard Lugert, Nurnberg (DE); Michael Rechinger, Nurnberg (DE)

Correspondence Address: LERNER AND GREENBERG, PA, P O BOX 2480, HOLLYWOOD, FL, 33022-2480, US

(73) Assignee: Faber-Castell AG

(21) Appl. No.: 10/903,113

(22) Filed: Jul. 30, 2004

(30) Foreign Application Priority Data
Dec. 19, 2003 (DE) 203 19 911.1

(51) Int. Cl. A61K 7/00

(52) U.S. Cl. 424/401

(57) ABSTRACT

A cosmetic solid lead is encased in a plastic sheath. The solid lead has a lipophilic phase that is liquid at 20° C. and that is present in an amount of at least 25%. The liphophilic phase is formed completely or at least predominantly from fatty alcohols having 16 to 20 carbon atoms. The solid lead is encased in a plastic based on a cellulose ester.

### BACKGROUND OF THE INVENTION

Field of the Invention

The invention relates to cosmetic solid leads, and in particular to a cosmetic solid lead encased with plastic and having a lipophilic phase that is liquid at room temperature or ambient temperature. The lipophilic phase is intimately mixed with a base material which is solid at ambient temperature.

The liquid phases used are conventionally mineral oils, such as paraffin oil with high contents of unbranched chains and fatty acid esters, such as isopropyl tetradecanoate (isopropyl myristate). The solid phases often form waxes and fatty acid derivatives often form the solid phase. The liquid lipophilic phase, which is often present in large amounts, brings about lead consistency, which allows soft marking on the skin and imparts shine to the areas of skwherein have been made up.

The liquid lipophilic phase, particularly when it is present in large amounts, brings about a reduction in the thermal stability of the cosmetic lead. At elevated temperatures, for example upon storage of the lead in heated rooms, the lead has a tendency to lose oil, i.e., the liquid lipophilic phase that had been previously mixed homogeneously with the solid phase separates out, possibly resulting in the formation of drops on the lead tip which can lead to soilings and interfere with application. This can be remedied to a certain extent by using a lead matrix with increased retention capacity for the liquid lipophilic phase, i.e., for example for oils. Microcrystalline waxes in particular are suitable. The described effects are also counteracted by using lead casings which absorb at least some of the exuded liquid phase. Materials which can do this are those with a microporous structure, for example wood or plastic to which pore formers have been added during preparation. However, liquid can only be absorbed if there is an open-pored structure, i.e., if the micropores open into the inner surface of the lead casing and are joined together. The latter can reduce the impermeability of the casing, which is undesired due to a consequent evaporation of volatile lead constituents. The micropores further hinder the production of clearly transparent casings or make it impossible altogether. A further way of countering the described effect consists in adding fillers with an open-pored structure to the casing. However, such fillers have considerable effects on the optical appearance of the casing and also influence its chemical and mechanical properties.

### SUMMARY OF THE INVENTION

It is accordingly an object of the invention to provide a solid cosmetic lead, which overcomes the above-mentioned disadvantages of the heretofore-known devices and methods of this general type and which provides for a cosmetic lead encased by plastic and having a high content of a lipophilic phase that is liquid at room temperature wherein loss of oil is avoided in an alternative way.

With the foregoing and other objects in view there is provided, in accordance with the invention, a cosmetic solid lead encased by plastic, comprising:
a lipophilic phase that is liquid at 20° C., that is present in an amount of at least 25%, and that is formed completely or at least predominantly from fatty alcohols having 16 to 20 carbon atoms; and
a plastic based on a cellulose ester.

In other words, the objects of the invention are achieved with the basic concept according to which the lipophilic phase, which is present in a large amount, at least 25%, is formed completely or at least predominantly from a chemical substance which dissolves in the plastic of the casing. This is very generally associated with the advantage that a plastic can be used which has an adequate absorption capacity for the liquid lipophilic phase, but which has to contain neither micropores nor fillers. The former harbors the risk of volatile lead constituents permeating. The latter impairs the transparency, for example, of plastics based on plasticized cellulose esters. For this group of plastics, in particular cellulose propionate (CP), cellulose acetate (CA) and cellulose acetobutyrate (CAB), it has surprisingly been found that fatty alcohols having 16 to 20 carbon atoms, in particular oleyl alcohol and/or isostearyl alcohol are suitable as lipophilic liquid phase or as its main constituent. These substances can be used to produce lead masses with a temperature resistance of up to at least 40° C. They are, moreover, able to dissolve within the cellulose ester, for example like a type of primary plasticizer or—looking at it another way—to dissolve the ester. Provided the lipophilic liquid phase is still mixed homogeneously with the lead matrix, the amount which crosses into the plastic is negligibly small. Only when the lipophilic phase emerges from the lead mass and forms a liquid film on the surface of the lead due to warming does it penetrate into the material layers of the casing close to the lead surface.

Leads with a consistency which is adequately solid at room temperature are obtained if the content of fatty alcohol is in the range from 25% to 65% and a content of wax and/or fatty acid derivative of from 5% to 35% is observed. Such leads withstand temperatures up to about 50° C. The dissolution capacity of, in particular, saturated branched fatty alcohols, specifically of isostearyl alcohol, is increased if adipic esters and/or citric esters (e.g. di-n-butyl adipate=Cetiol B or acetyl tributylcitrate), known as plasticizers for cellulose esters, are added to the lead mass. These substances also improve the thermal stability of the lead mass irrespective of the oil absorption capacity of the casing. Stabilization and improvement of the oil retention ability is, as already mentioned above, achieved by microcrystalline waxes (paraffin waxes), or else by hydrogenated castor oil. The formulations according to the invention are further wherein by low filler contents (organic or inorganic fillers), or fillers may be dispensed with completely. The cellulose ester of the casing preferably has a plasticizer content of from 5 to 25%, in particular from 18 to 22%. This ensures adequate mechanical stability, but at the same time also good sharpenability and an adequate absorption capacity for a lipophilic phase which secretes from the lead mass.

To produce an encased cosmetic lead or a stick having a plastic casing, the lead masses, which are homogenized and molten at 70° C. to 100° C., are poured directly into a tubular plastic casing or, after mixing the raw materials, strands of lead are extruded and introduced into a plastic casing.

Other features which are considered as characteristic for the invention are set forth in the appended claims.

Although the invention is illustrated and described herein as embodied in a cosmetic solid lead, it is nevertheless not intended to be limited to the specific exemplary details, since various modifications and structural changes may be made therein without departing from the spirit of the invention and within the scope and range of equivalents of the claims.

The construction and method of operation of the invention, however, together with additional objects and advantages thereof will be best understood from the following description of specific examples that represent embodiments of the invention.

Base Formulation

| | |
|---|---|
| Unsaturated unbranched and/or | 25-65% |
| saturated, branched fatty alcohol | |
| Waxes and fatty acid derivatives solid at 40° C. | 5-35% |
| Adipic esters and/or citric esters | 0-12% |
| Pigments | 0-30% |
| Fillers | 0-20% |
| Additives, such as thickeners, antioxidants, | 2-15% |
| preservatives, emulsifiers, etc. | |

### EXAMPLE 1

| Hydrogenated castor oil | | 10.00% |
|---|---|---|
| Stearic acid | | 5.00% |
| Carnauba wax | | 3.50% |
| Oleyl alcohol (Novol) | | 30.00% |
| Isostearyl alcohol (CAS No. 27458-93-1) | | 20.00% |
| Acetyl tributylcitrate (CAS No. 77-90-7) | | 2.00% |
| Thickener (Bentone 27) | | 2.45% |
| Preservative (Sensiva SC50) | | 0.33% |
| Antioxidant (ascorbyl palmitate) | | 0.02% |
| Filler (talc) | | 8.00% |
| Perfume | | 0.50% |
| Pigments: | titanium dioxide | 4.00% |
| | Cloisonne Red | 14.20% |

### EXAMPLE 2

| Oleyl alcohol (Novol) | | 54.38% |
|---|---|---|
| Microcrystalline wax (CAS No. 63231-60-7) | | 11.89% |
| Candelilla wax | | 1.70% |
| Fillers | (Spherica) | 3.40% |
| | Covabead PMMA | 3.40% |
| | CAB-O-SIL | 1.13% |
| Pigments: | Cloisonne Red | 7.36% |
| | Timiron Diamond Cluster MP 149 | 6.80% |
| | Timiron Supersheen MP 1001 | 5.66% |
| | Timica Brilliant White | 2.04% |
| | Cloisonne Gold | 0.68% |
| Additives: | Bentone 27 V | 1.13% |
| | Arconate HP | 0.37% |
| | Decanox MTS 70 | 0.05% |
| | Ascorbyl palmitate | 0.01% |

### EXAMPLE 3

| Isostearyl alcohol (CAS No. 27458-93-1) | | 40.49% |
|---|---|---|
| Microcrystalline wax (CAS No. 63231-60-7) | | 15.75% |
| Candelilla wax | | 2.25% |
| Carboxylic ester (Cetiol CC) | | 11.25% |
| Pigments: | EA 1156 | 3.37% |
| | C 47-060 | 0.17% |
| | C 33-5000 | 1.12% |
| | Cloisonnene Gold | 0.56% |
| | Timica Brilliant White | 6.75% |
| | Timiron Gold MP 127 | 9.00% |
| Additives: | Cetiol B | 4.50% |
| | Decanox MTS 70 | 3.37% |
| | Ascorbyl palmitate | 1.12% |
| | Bentone 27 V | 0.06% |
| | Arconate HP | 0.02% |
| | Elestab CPN | 0.11% |
| | Sensiva SC50 | 0.11% |

### EXAMPLE 4

| Isostearyl alcohol (CAS No. 27458-93-1) | | 38.71% |
|---|---|---|
| Microcrystalline wax (CAS No. 63231-60-7) | | 14.65% |
| 1-Propylene, 2-methyl-, homopolymer | | 13.60% |
| (C4H8) × polyisobutylene (Rewopal IB1000) | | |
| Candelilla wax | | 2.09% |
| Pigments: | C 19-003 | 0.42% |
| | C 33-5000 | 0.42% |
| | Cloisonnene Red | 13.08% |
| | Flamenco Superpearl | 4.19% |
| | Cloisonnene Orange | 4.19% |
| Additives: | Cetiol B | 6.28% |
| | Decanox MTS 70 | 0.06% |
| | Ascorbyl palmitate | 0.02% |
| | Bentone 27 V | 1.57% |
| | Arconate HP | 0.52% |
| | Elestab CPN | 0.10% |
| | Sensiva SC50 | 0.10% |

All of the above percentages are percentages by weight.

The following is a list of raw material suppliers from which the above-mentioned materials are available:

| | |
|---|---|
| Bentone 27 V: | Elementis, B-9000 Gent, Belgium |
| Sensiva SC50: | Schülke & Mayr, D-22851 |
| | Norderstedt, Germany |
| Cloisonnene Red: | Engelhard, Iselin, NJ. 08830 |
| Cloisonnene Gold: | Engelhard, Iselin, NJ. 08830 |
| Cloisonnene Orange: | Engelhard, Iselin, NJ. 08830 |
| Timica Brilliant White: | Engelhard, Iselin, NJ. 08830 |
| Flamenco Superpearl: | Engelhard, Iselin, NJ. 08830 |
| Spherica: | Ikeda, Tokyo 100-0005, Japan |
| Cetiol B: | Cognis, D-40551 Düsseldorf, Germany |
| Covabead PMMA: | LCW, F-95310 Saint-QuenI‘aumone, |
| | France |
| CAB-O-SIL TS 610: | Sun Chemical, Cincinnati, OH 45232 |
| C 47-060: | Sun Chemical, Cincinnati, OH 45232 |
| C 33-5000: | Sun Chemical, Cincinnati, OH 45232 |
| C 19-003: | Sun Chemical, Cincinnati, OH 45232 |
| Decanox MTS 70: | ADM, Decatur, IL 62526 |
| Ascorbyl palmitate: | Hoffman La Roche, CH-4070 Basel, |
| | Switzerland |
| EA 1156: | Color Techniques, South Plainfield, NJ |
| Timiron Gold MP 127: | Merck, D-64271 Darmstadt, Germany |
| Timiron Diamond | |
| Cluster MP149: | Merck, D-64271 Darmstadt, Germany |
| Timiron Supersheen | |
| MP-1001: | Merck, D-64271 Darmstadt, Germany |
| Elestab CPN: | Laboratoires, F-54425 Pulnoy, France |
| Arconate HP: | Lyondell Chemie, NL-3014 Rotterdam, |
| | Netherlands |
| Rewopal PIB 1000: | WITICO Surfactants GmbH, |
| | D-36396 Steinau, Germany |
| Novol: | Croda, D-41334 Nettetal, Germany |

This application claims the priority, under 35 U.S.C. § 119, of German patent application No. 203 19 911.1, filed Dec. 19, 2003; the entire disclosure of the prior application is herewith incorporated by reference.

1. A cosmetic solid lead encased by plastic, comprising:
a lipophilic phase that is liquid at 20° C., that is present in an amount of at least 25%, and that is formed completely or at least predominantly from fatty alcohols having 16 to 20 carbon atoms; and
a plastic based on a cellulose ester.

2. The solid lead according to claim 1, which comprises a component of oleyl alcohol.

3. The solid lead according to claim 1, which comprises a component of isostearyl alcohol.

4. The solid lead according to claim 1, having a fatty alcohol content of from 25% to 65%.

5. The solid lead according to claim 1, which comprises at least one of an adipic ester and a citric ester.

6. The solid lead according to claim 5, having an ester content of from 0.1% to 12%.

7. The solid lead according to claim 1, which comprises at least one wax and/or at least one fatty acid derivative that are solid at 20° C.

8. The solid lead according to claim 7, which comprises a microcrystalline wax.

9. The solid lead according to claim 7, having a content of at least one of wax and fatty acid from 5% to 35%.

10. The solid lead according to claim 1 encased in a cellulose ester selected from the group consisting of cellulose acetate, cellulose propionate, and cellulose acetobutyrate.

11. The solid lead according to claim 1, which comprises a plasticizer content from 5 to 25%.

12. The solid lead according to claim 11, wherein the plasticizer content is from 18 to 22%.
